(19) United States
(12) Patent Application Publication
(10) Pub. No.: US 2001/0009671 A1
(43) Pub. Date: Jul. 26, 2001

(54) COSMETIC COMPOSITIONS

(76) Inventor: EVELYN MARIANNE HELBICHE NEE FROHNE, ALSBACH (DE)

Correspondence Address: A E MATTHEWS, THE PROCTER & GAMBLE COMPANY PATENT DIV, WINTON HILL TECHNICAL CENTER, 6083 CENTER HILL AVENUE, CINCINNATI, OH, 45224

(21) Appl. No.: 09/194,265

(22) PCT Filed: May 13, 1997
(86) PCT No.: PCT/US97/08053

§ 371 Date: None

(30) Foreign Application Priority Data
May 22, 1996 (GB) 9610670.3

(51) Int. Cl. A61K 9/00

(52) U.S. Cl. 424/400

(57) ABSTRACT

The invention relates to a cosmetic composition comprising: (i) greater than 2% of a panthenol oil regulating agent; and (ii) from 0.1 % to 10% of a particulate, oil-absorbing polymer, and (iii) from 20% to 97.9% of a cosmetically acceptable carrier. The compositions, which preferably take the form of oil-in-water emulsions, provide both immediate and long-term control of oily and/or shiny skin.

### TECHNICAL FIELD

The present invention relates to leave-on, skin care cosmetic compositions which are useful for moderating and regulating the oily and/or shiny appearance of skin.

### BACKGROUND OF THE INVENTION

In the field of skin care compositions, the population is often classified by skin type, e.g., dry, normal, oily, combined dry/normal, combined dry/oily, or combined normal/oily skin. The latter two classes are hereinafter alternatively referred to as “combined skin” and are typified by areas of the facial skin, especially the nose and forehead (often referred to as the “T-zone”) which exhibit a more oily appearance relative to other areas of the face, e.g. the cheeks, remain dry.

Persons with an oily skin type or combined skin type typically manifest an oily and/or shiny skin appearance between cleansings. This oily or shiny appearance generally increases as the day progresses following cleansing of the skin. In order to avoid such appearance, individuals must throughout the day either cleanse the skin, blot the skin, apply oil absorbing powders to the skin, or take some other measure to minimise the appearance of oil or shine.

Therefore, it has been desired in the art to provide topical compositions such as lotions and make-ups which minimise the appearance of oil and/or shine on the skin, especially oily or combined skin. Several topical compositions which are said to be designed for controlling oily and/or shiny skin are known in the art. For example, facial moisturisers and make-up said to have such property are known.

While certain formulations have been designed in an attempt to control the oily and/or shiny appearance of skin, there remains a need to provide improved topical compositions for minimising the appearance of skin oil and/or shine. In addition to minimising oil and/or shine, such compositions should not unacceptably discolour the skin. There is a particular need to provide improved facial compositions which minimise the appearance of skin oil and/or shine, provide and maintain an even (i.e., uniform coverage) complexion and acceptable skin tone for extended periods after application, and/or which have extended wear resistance after application.

EP-A-392,426 describes an emulsifier-free hand and body lotion comprising cross-linked hydrophobic methacrylate copolymers with an active dispersed within the polymer. The document provides a review of some of the background art relating to absorbent polymer beads.

WO 95/09598 describes a liquid make-up composition which can utilise the same cross-linked hydrophobic acrylate or methacrylate copolymers. The copolymers are said to be valuable for reducing shine and controlling oil while helping to provide effective moisturisation benefits. The compositions described can also optionally contain a moisturiser which can be e.g. panthenol, pyridoxine or Vitamin B complex.

The B vitamins or vitamin B complex have heretofore further been used to treat a number of conditions. For example, the following compounds have had the respective applications: riboflavin (acne, diabetes, anti-oxidant therapy, anaemia, skin disorders, stress); nicotinic acid (atherosclerosis, pellegra, high cholesterol, high blood pressure, skin inflammation, antioxidant therapy); nicotinamide (pellagra, skin inflammation, anti-oxidant therapy); pantothenic acid (acne, anaemia, arthritis, high cholesterol, atherosclerosis, alcohol detoxification, infections, hair loss); pyridoxine (acne, anaemia, high cholesterol, skin inflammation, immune disorders, antioxidant therapy, carpal tunnel syndrome, pre-menstrual syndrome). These utilities and those of other B vitamin (complex) compounds are further described, along with a discussion of their contraindications and deficiency symptoms, in The Doctor's Vitamin and Mineral Encyclopedia, Hendler, S. S., pp. 49-82 (Simon & Schuster, New York 1990) and Nutrients Catalog, Newstrom, H., pp. 11-90 (McFarland & Co. 1993).

It is disclosed in pending U.S. application Ser. No. 08/554067 that certain B group vitamins, including panthenol, have recently been found to be useful as active agents for regulating the oily and/or shiny appearance of mammalian without unacceptably discolouring the skin, e.g. by unacceptable skin flushing or reddening.

As discussed above, panthenol is useful for controlling the oily and/or shiny appearance of skin, however it remains desirable to improve the overall oil regulating effect, particularly soon after its application to the skin. It has been found that topical compositions containing both the panthenol oil regulating agent and certain oil-absorbing polymers both provide an immediate reduction in shine and oily appearance, minimise the appearance of skin oil and/or shine over time, provide and maintain substantially uniform coverage and an acceptable skin tone for extended periods after application, and/or have extended wear resistance after application.

It is an object of the present invention to provide topical compositions for regulating the oily and/or shiny appearance of mammalian skin, especially facial skin.

It is a further object of this invention to provide such topical compositions which provide an immediate reduction in shine and oily appearance, regulate the appearance of oily and/or shiny mammalian skin over time, provide and maintain substantially uniform coverage for extended periods after application to the skin, provide and maintain an acceptable skin tone for extended periods after application to the skin, and/or have extended wear resistance after application to the skin.

Other objects of the subject invention will be apparent from the disclosure which follows.

### SUMMARY OF THE INVENTION

The present invention relates to a leave-on, skin care cosmetic composition comprising:

i) greater than 2% of a panthenol oil regulating agent; and

ii) from 0.1% to 10% of a particulate, oil-absorbing polymer; and

iii) from 20% to 97.9% of a cosmetically acceptable carrier.

It has surprisingly been found that such compositions are especially useful for regulating the appearance of oily and/or shiny skin, including oily and combined skin.

All percentages and ratios herein are by weight of the composition, unless otherwise indicated.

### DETAILED DESCRIPTION OF THE INVENTION

The compositions of the present invention are useful as topical compositions, i.e., they are suitable for administration to the surface of the skin of a biological subject such as a mammal. The compositions of the subject invention are administered topically to a biological subject, i.e., by the direct laying on or spreading of the composition on the skin of the subject.

As used herein, “cosmetically acceptable” means that a material (e.g., compound or composition) which the phrase describes is suitable for use in contact with the tissues of humans and lower animals without undue toxicity, incompatibility, instability, irritation, allergic response and the like.

As used herein, “regulating the oily and/or shiny appearance of skin” means preventing, retarding and/or arresting the appearance of oil and/or shine on the skin. By regulating the oily and/or shiny appearance of the skin, one or more of the following benefits are achieved: there is a noticeable decrease in the visible oil, shine, or highlights on the skin; the skin is substantially free from visible oiliness, shine, or highlights; the skin has a substantially matte finish; the user has a more uniform complexion. Regulating the oily and/or shiny appearance of the skin may result in more uniform and lasting coverage of the skin by the composition, increased wear resistance of the composition and/or a decrease in the incidence or severity of skin oil breaking through the composition so as to become visibly apparent.

In general, the compositions of the present invention are in the form of an aqueous emulsion, preferably an oil-in-water emulsion. Suitably, the pH is in the range from about 4 to about 8, more preferably from about 5 to about 8 and especially from about 6 to about 8.

Panthenol Oil Regulating Agent

The compositions of this invention comprise as an essential ingredient at least 2% of a panthenol oil regulating agent. As used herein, “panthenol oil regulating agent” means panthenol, a C₁ - C₆ alkyl ester or ether derivative thereof, such as panthenyl ethyl ether, or mixtures thereof.

The panthenol oil regulating agent leads to a long-lasting reduction in the oiliness or oily appearance of the skin.

Preferably, the panthenol oil regulating agent is panthenol itself, more preferably D-panthenol. The composition preferably comprises from about 2.5% to about 20%, more preferably from about 3% to about 10%, even more preferably from about 3% to about 5% by weight, of the panthenol oil regulating agent.

Although it is contemplated that other oil regulating agents may be used in addition to the panthenol oil regulating agent described herein, the compositions herein can comprise an oil regulating agent consisting or consisting essentially of panthenol.

Oil-Absorbing Polymer

The compositions of the present invention also comprise a particulate, oil-absorbing polymer. This polymer is particularly valuable for providing an immediate reduction in shine and oily appearance. It can further help to provide effective moisturisation benefits. As used herein, “particulate” means that the polymer is available in the form of a free-flowing powder and “oil-absorbing” means that the powder can absorb at least 50%, preferably at least 100% and more especially at least 200% of its weight of olive oil and remain free-flowing. In general, oil-absorbing polymers according to the invention are insoluble in water and do not swell significantly therein, although they are preferably dispersible in water. The particulate, oil-absorbing polymer is used in an amount of from about 0.1% to about 10%, preferably from about 0.3% to about 5%, more preferably from about 1% to about 3% by weight.

Preferred oil-absorbing polymers for use herein are hydrophobic in character such as acrylate or methacrylate polymers or copolymers. Such hydrophobic polymers are able to preferentially absorb oils in the presence of other liquids.

In especially preferred embodiments the oil-absorbing polymer is a cross-linked copolymer. Suitable cross-linking agents are well know in the art and include, for example, (1) compounds having at least two polymerisable double bonds; (2) compounds having at least one polymerisable double bond and at least one functional group reactive with the acid-containing monomer material; (3) compounds having at least two functional groups reactive with the acid-ontaining monomer material; and (4) polyvalent metal compounds which can form ionic cross-linkages.

Cross-linking agents having at least two polymerisable double bonds include (i) di- or polyvinyl compounds such as divinylbenzene and divinyltoluene; (ii) di- or poly-esters of unsaturated mono- or poly-carboxylic acids with polyols including, for example, di- or triacrylic acid esters of polyols such as ethylene glycol, trimethylol propane, glycerine, or polyoxyethylene glycols; (iii) bisacrylamides such as N,N-methylenebisacrylamide; (iv) carbamyl esters that can be obtained by reacting polyisocyanates with hydroxyl group-containing monomers; (v) di- or poly-allyl ethers of polyols; (vi) di- or poly-allyl esters of polycarboxylic acids such as diallyl phthalate, diallyl adipate, and the like; (vii) esters of unsaturated mono- or poly-carboxylic acids with mono-allyl esters of polyols such as acrylic acid ester of polyethylene glycol monoallyl ether; and (viii) di- or tri-allyl amine.

Cross-linking agents having at least one polymerisable double bond and at least one functional group reactive with the acid-containing monomer material include N-methylol acrylamide, glycidyl acrylate, and the like. Suitable cross-linking agents having at least two functional groups reactive with the acid-containing monomer material include glyoxal; polyols such as ethylene glycol and glycerol; polyamines such as alkylene diamines (e.g., ethylene diamine), polyalkylene polyamines, polyepoxides, di- or polyglycidyl ethers and the like. Suitable polyvalent metal cross-linking agents which can form ionic cross-linkages include oxides, hydroxides and weak acid salts (e.g., carbonate, acetate and the like) of alkaline earth metals (e.g., calcium, magnesium) and zinc, including, for example, calcium oxide and zinc diacetate.

Cross-linking agents of many of the foregoing types are described in greater detail in Masuda et al., U.S. Pat. No. 4,076,663, issued Feb. 28, 1978, and Allen et al., U.S. Pat. No. 4,861,539, issued Aug. 29, 1989, both incorporated herein by reference. Preferred cross-linking agents include the di- or polyesters of unsaturated mono- or polycarboxylic acids mono-allyl esters of polyols, the bisacrylamides, and the di- or tri-allyl amines. Specific examples of especially preferred cross-linking agents include N,N′-methylenebisacrylamide and trimethylol propane triacrylate.

The cross-linking agent will generally constitute from about 0.01 mole percent to 50 mole percent of the resulting oil-absorbing copolymer. More generally, the cross-linking agent will constitute from about 0.1 mole percent to 30 mole percent of the oil-absorbing copolymer used herein.

The oil-absorbing polymer is preferably in the form of a polymer lattice. Alternatively, the hydrophobic polymer can take the form of a porous particle having a surface area (N₂-BET) in the range from about 50 to 500, preferably 100 to 300 m²/g.

Preferred polymer materials are in the form of a powder, the powder being a combined system of particles. The system of powder particles forms a lattice which includes unit particles of less than about one micron in average diameter, agglomerates of fused unit particles of sized in the range of about 20 to 100 μm in average diameter and aggregates of clusters of fused agglomerates of sizes in the range of about 200 to 1,200 μm in average diameter.

A preferred copolymer has the structural formula:

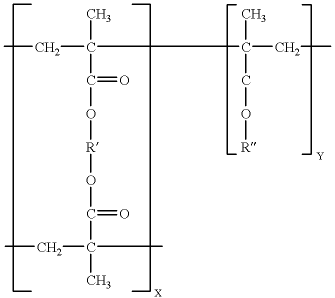

where the ratio of x to y is from 1:99 to 99:1, R′ is (—CH₂CH₂—)ₐ and R″ is —(CH₂)_{b}CH₃, where a is from 1 to 8 and b is from 0 to 29. Preferably, x:y is about 80:20, a is 1 and b is 0 or 11.

Suitable materials of this type are manufactured by the Dow Corning Corporation, Midland, Mich., USA, and sold under the name Polytrap®. Polytrap® is an ultralight free-flowing white powder and the particles are capable of absorbing high levels of lipophilic liquids and some hydrophilic liquids while at the same time maintaining a free-flowing powder character. The Polytrap® powder structure consists of a lattice of unit particles less than one micron that are fused into agglomerates of 20 to 100 microns and the agglomerates are loosely clustered into macro-particles or aggregates of about 200 to about 1200 micron size. The copolymer powder is capable of containing as much as four times its weight of fluids, emulsions, dispersions or melted solids. Preferred is Polytrap® Q5-6603.

The copolymer can optionally have an active ingredient absorbed therein, preferably dispersed uniformly throughout and entrapped within the copolymer lattice. The active ingredient can be the panthenol oil regulating agent as hereinbefore described, skin compatible humectants, emollients, moisturising agents and sunscreens. Preferably the active ingredient is selected from the panthenol oil regulating agent, skin compatible humectants and mixtures thereof. Adsorption of active ingredients onto the copolymer powder can be accomplished using a stainless steel mixing bowl and a spoon, wherein the active is added to the powder and the spoon is used to gently fold the active into the copolymer powder. Low viscosity fluids may be adsorbed by addition of the fluids to a sealable vessel containing the copolymer and then tumbling the materials until a consistency is achieved. More elaborate blending equipment such as ribbon or twin cone blenders can also be employed. Preferred active ingredients for use herein include glycerine and mixtures of glycerin with panthenol. Preferably, the weight ratio of active ingredient:carrier is from about 1:4 to about 5:1, more preferably from about 1:1 to about 4:1.

In a preferred embodiment 1 part of the copolymer is loaded with from about 3 parts to about 4 parts of an aqueous solution comprising from about 30% to about 50% glycerine, from about 5% to about 10% panthenol and from about 0.5% to about 0.7% xanthan gum by weight of the solution. Without wishing to be bound by theory, it is believed that pre-loading the copolymer with glycerine and panthenol mitigates its hydrophobicity so that it can be more readily dispersed in an aqueous phase. In this manner, emollient oils can be incorporated in the oil phase of an emulsion comprising the copolymer in an aqueous phase without substantially interfering with the oil absorbing capacity of the copolymer. When the emulsion is spread on the skin the copolymer is able to preferentially exchange the adsorbed glycerin and panthenol for excess skin oils.

Also suitable as a highly cross-linked polymethacrylate copolymer is Microsponges® 5647. This takes the form of generally spherical particles of cross-linked hydrophobic copolymer having a pore size of from about 0.01 to about 0.05 μm and a surface area of 200-300 m²/g. Again, it is preferably loaded with an active ingredient in the levels described above.

Cosmetically Acceptable Carrier

The compositions of the present invention further include from 20% to 97.9%, preferably from 40% to 97.9% of a cosmetically acceptable carrier. The phrase “cosmetically acceptable carrier”, as used herein, means one or more compatible solid or liquid fillers the like, which are cosmetically acceptable as defined herein. The term “compatible”, as used herein, means that the components of the compositions of this invention are capable of being comingled with the oil regulating agents and copolymers of the present invention, and with each other, in a manner such that there is no interaction which would substantially reduce the efficacy of the composition under ordinary use situations. Non-limiting example of such carriers include mica and water. Preferably the filler does not comprise significant quantities of liquid oils that would be absorbed by the polymer and impair its efficacy. Preferably the carrier is water.

Optional Components

The compositions of the present invention can additionally comprise a wide variety of optional compatible components. Typical of such optional components are:

Thickening Agents

The compositions of the present invention can comprise a thickening agent, especially a nonionic, polymeric thickening agent. Suitable examples include the mixture of polyacrylamide and C₁₃₋₁₄ isoparaffin and laureth-7, available as Sepigel 305 from Seppic Corp., Fairfield, N.J.; carbomers such as Carbopol 954 from B.F. Goodrich; branched polysaccharides such as scleroglucan available under the tradename Clearogel® CS 11 from Michel Mercier Products Inc., Mountainside, N.J.); gums (e.g. guar gum, xanthan gum and the like) and cellulose derivatives such as cetyl hydroxyethyl cellulose. These nonionic, polymeric thickening agent are suitably used at levels from about 0.05% to about 5%, preferably from about 0.1% to about 3%, and most preferably from about 0.2% to about 2% by weight of the composition.

Humectants

Another optional component of the compositions of the present invention is a humectant. When used herein, the humectant may suitably be used at levels from about 0.1% to about 20%, more preferably from about 2% to about 15%, and most preferably from about 4% to about 12% by weight of the composition. Even though these materials are defined herein as humectants, they can also possess moisturising, skin conditioning, and other related properties.

Examples of humectants useful herein include materials such as urea; guanidine; aloe vera in any of its variety of forms (e.g. aloe vera gel); polyhydroxy alcohols such as sorbitol, glycerine, low molecular weight polypropylene glycols (e.g., dipropylene glycol and tripropylene glycol), hexanetriol, propylene glycol, butylene glycol, hexylene glycol, and the like; polyethylene glycol; sugars and starches; sugar and starch derivatives (e.g., alkoxylated glucose); hyaluronic acid; chitin; lactamide monoethanolamine; acetamide monoethanolamine; propoxylated glycerol (as described in U.S. Pat. No. 4,976,953 to Orr et al., issued Dec. 11, 1990); and mixtures thereof.

Preferred humectants include those materials selected from the group consisting of urea, propylene glycol, pentane-1,2-diol, 1,3-dihydroxypropane, glycerine, propoxylated glycerine, glyceryl polyacrylate, butylene glycol, hexylene glycol, 1,4-dihydroxyhexane, 1,2,6-hexanetriol, dipropylene glycol, tripropylene glycol, and mixtures thereof. More preferred are those selected from the group consisting of propylene glycol, pentane-1,2-diol, urea, glycerine, glyceryl polyacrylate and mixtures thereof.

Emollients

The compositions of the present invention may also include an emollient. Examples of suitable emollients include, but are not limited to, volatile and non-volatile silicone oils (e.g., dimethicone, cyclomethicone, dimethiconol, and the like); non-polar fatty acid and fatty alcohol esters (e.g. cetearyl isononanoate); vegetable oils; hydrocarbons (e.g. isohexadecane), and mixtures thereof. The emollient oils are suitably chosen so that they are readily absorbed by the skin and do not themselves contribute to its oily and/or shiny appearance.

Suitable total levels of emollients are from about 0.1% to about 25%, more preferably from about 1% to about 15%, and most preferably from about 2% to about 10% by weight of the composition.

Sunscreens

The compositions of the present invention may also include organic sunscreens. Non-limiting examples of suitable sunscreens include octyl methoxycinnamate, butyl methoxydibenzoylmethane, 4-methylbenzylidenecamphor, octocrylene and mixtures thereof.

Suitable total levels of sunscreens are from about 0.1% to about 15%, more preferably from about 1% to about 10%, and most preferably from about 2% to about 8% by weight of the composition.

Additional Ingredients

A variety of additional ingredients can be incorporated into the compositions of the present invention. Non-limiting examples of these additional ingredients include vitamins and derivatives thereof (e.g. tocopherol and the like); waxes (both naturally occurring and synthetic); polymers for aiding the film-forming properties and substantivity of the composition (such as a copolymer of hexadecane and vinyl pyrrolidone, an example of which is available from ISP Investment as Antaron V-216); preservatives for maintaining the antimicrobial integrity of the compositions; skin penetration aids such as DMSO, 1-dodecylazacycloheptan-2-one (available as Azone® from the Upjohn Co.) and the like; skin bleaching (or lightening) agents including but not limited to hydroquinone, kojic acid and sodium metabisulfite; chelators and sequestrants; and aesthetic components such as fragrances, pigments, colourings, essential oils, skin sensates, astringents, skin soothing agents, skin healing agents and the like, nonlimiting examples of these aesthetic components include aloe vera, clove oil, menthol, camphor, eucalyptus oil, eugenol, menthyl lactate, witch hazel distillate, allantoin, bisabolol, dipotassium glycyrrhizinate and the like.

The following Examples further describe and demonstrate the preferred embodiments within the scope of the present invention. Polytrap® Q5-6603 is an oil-absorbing polymer according to the invention.

### EXAMPLE I

The following is a representative skin cream according to the invention which is in the form of an oil-in-water emulsion having a pH in the range from 6.5 to 7, The Polytrap® is pre-loaded with 0.5% by weight of the composition of the panthenol, 2.5% by weight of the composition of the glycerine and with the xanthan gum as a 2% aqueous solution. The balance of the panthenol is disolved in the aqueous phase prior to emulsification. The pre-loaded Polytrap® is dispersed in the emulsion formed from the other ingredients.

| Ingredient | % |
|---|---|
| Water | to 100% |
| Glycerin | 6.00 |
| Panthenol | 3.00 |
| Phenyl trimethicone | 3.00 |
| Polytrap ® Q5-6603 | 2.00 |
| PVP/hexadecene copolymer | 2.00 |
| Propylene glycol | 1.81 |
| Dimethicone | 1.44 |
| Behenyl alcohol | 1.40 |
| Butylene glycol | 1.00 |
| Isopropyl palmitate | 1.00 |
| Triethanolamine | 0.80 |
| Potassium cetyl phosphate | 0.40 |
| Carbopol 954 | 0.30 |
| Acrylates/C₁₀₋₃₀ alkyl acrylate | 0.20 |
| crosspolymer | |
| Perfume | 0.20 |
| Disodium EDTA | 0.10 |
| Preservatives | 1.07 |
| Xanthan gum | 0.07 |
| Dimethiconol | 0.06 |
| Plant extracts | 0.38 |

### EXAMPLE II

The following is a representative oil-in-water make-up composition according to the invention. The make-up is in the form of an oil-in-water emulsion having a pH in the range from 7 to 8. The panthenol and Polytrap are incoprorated as described for Example I.

| Ingredient | % |
|---|---|
| Water | to 100% |
| Glycerin | 6.00 |
| Cetearyl Isononanoate | 4.00 |
| Mineral Oil | 4.00 |
| Propylene Glycol | 3.52 |
| Panthenol | 3.00 |
| Octyl Methoxycinnamate | 2.50 |
| Polytrap ® Q5-6603 | 2.00 |
| Lanolin oil | 2.00 |
| Glyceryl stearate | 1.80 |
| Propylene glycol stearate | 1.80 |
| PEG-40 stearate | 1.40 |
| Palmitic acid | 1.00 |
| PVP/hexadecane copolymer | 1.00 |
| Stearic acid | 1.00 |
| Tocopheryl acetate | 1.00 |
| Dimethicone | 0.60 |
| Hectorite | 0.40 |
| Perfume | 0.30 |
| Preservatives | 1.07 |
| Sodium Hydroxide | 0.13 |
| Xantham Gum | 0.13 |
| Plant extracts | 0.380 |
| Ethoxydiglycol | 0.04 |
| Antioxidant | 0.04 |
| Pigments | 7.661 |

The cosmetic compositions of Examples I and II display good short and long-term oil control together with excellent stability and in-use performance characteristics.

1. A cosmetic composition comprising:
i) greater than 2% of a panthenol oil regulating agent; and
ii) from 0.1% to 10% of a particulate, oil-absorbing polymer; and
iii) from 20% to 97.9% of a cosmetically acceptable carrier.

2. A cosmetic composition according to claim 1 wherein the composition comprises from about 2.5% to about 20%, more preferably from about 3% to about 10%, even more preferably from about 3% to about 5% of the panthenol oil regulating agent.

3. A cosmetic composition according to any one of claims 1 or 2 wherein the panthenol oil regulating agent is panthenol.

4. A cosmetic composition according to claim 1 wherein the composition comprises from about 0.3 % to about 5%, more preferably from about 1% to about 3% of the oil-absorbing polymer.

5. A cosmetic composition according to any one of claims 1 to 4 wherein the oil-absorbing polymer is a a cross-linked acrylate or methacrylate hydrophobic copolymer having the general formula 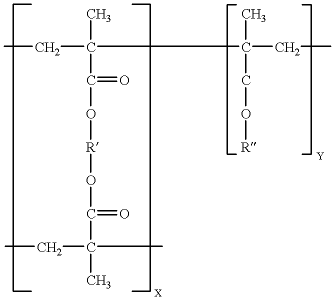
where the ratio of x to y is from 1:99 to 99:1, R′ is (—CH₂CH₂—)ₐ and R″ is —(CH₂)_{b}CH₃, where a is from 1 to 8 and b is from 0 to 29.

6. A cosmetic composition according to claim 5 wherein x is about 80:20, a is 1 and b is 0 or 11.

7. A cosmetic composition according to any one of claims 5 or 6 wherein the copolymer has an active ingredient absorbed upon it in a weight ratio of from about 1:4 to about 5:1, preferably from about 1:1 to about 4:1.

8. A cosmetic composition according to any one of claims 1 to 7 wherein the composition is an emulsion composition having a pH in the range from about 4 to about 8, preferably from about 4 to about 7 and especially from about 4.5 to about 6.

9. A cosmetic composition according to any one of claims 1 to 8 wherein the composition is an oil-in-water emulsion.
